(19) United States
(12) Patent Application Publication
(10) Pub. No.: US 2021/0251606 A1
(43) Pub. Date: Aug. 19, 2021

(54) ULTRASONIC DIAGNOSTIC APPARATUS, OPERATING METHOD OF ULTRASONIC DIAGNOSTIC APPARATUS, AND STORAGE MEDIUM

(71) Applicant: Konica Minolta, Inc., Tokyo (JP)

(72) Inventors: Makiko URABE, Yokohama-shi (JP); Yoshihiro TAKEDA, Tokyo (JP); Akihiro KAWABATA, Tokyo (JP)

(21) Appl. No.: 17/167,414

(22) Filed: Feb. 4, 2021

(30) Foreign Application Priority Data
Feb. 13, 2020 (JP) 2020-021997

(51) Int. Cl. A61B 8/00; A61B 8/08; A61B 5/00; H04N 5/232

(52) U.S. Cl. CPC A61B 8/463; A61B 8/5261; A61B 5/0035; A61B 8/4405; A61B 5/0077; H04N 5/23229; A61B 5/7425

(57) ABSTRACT

An ultrasonic diagnostic apparatus includes an ultrasonic image generator that generates ultrasonic image data of a moving image based on a reception signal received from an ultrasonic probe. The ultrasonic probe transmits and receives an ultrasonic wave to and from a subject. The ultrasonic diagnostic apparatus further includes a camera image generator that takes an image of an imaging target and generates camera image data of a moving image. The ultrasonic diagnostic apparatus further includes a hardware processor that performs, in response to operation input related to ultrasonic diagnosis, processing related to the camera image data.

### CROSS-REFERENCE TO RELATED APPLICATIONS

The entire disclosure of Japanese Patent Application No. 2020-021997 filed on Feb. 13, 2020 is incorporated herein by reference in its entirety.

### BACKGROUND

### Technological Field

The present invention relates to an ultrasonic diagnostic apparatus, an operating method of an ultrasonic diagnostic apparatus, and a storage medium.

### Description of Related Art

In ultrasonic diagnosis, an ultrasonic image representing the state of a heart, an unborn baby, and the like can be obtained by a simple operation of attaching an ultrasonic probe to the body surface or the inside of the body cavity of a patient as a subject. This operation is also highly safe and therefore can be performed repeatedly. There is known an ultrasonic diagnostic apparatus used for performing such ultrasonic diagnosis.

In orthopedic treatment or rehabilitation, a motion range of a patients joint is checked. At this time, it is possible to improve the efficiency of medical treatment or rehabilitation by using an ultrasonic diagnostic apparatus and a camera in order to record, edit, and display a live image of the camera and an ultrasonic image. For example, before rehabilitation, a physiotherapist checks a camera image taken at the time of medical treatment and an ultrasonic image at the time of examination. As a result, even when there is no detailed communication from the doctor, the physiotherapist can easily recognize the location of the affected portion and the degree of the disease and work efficiently. Camera images are thus useful.

There is known an ultrasonic diagnostic apparatus that generates a camera image and an ultrasonic image. For example, in such an ultrasonic diagnostic apparatus, a probe position detector detects a position of an ultrasonic probe, a camera moves to, turns to, and zooms in the detected position, and a still camera image and a still ultrasonic image are generated, combined, and displayed (see JP 2009-207800 A).

Furthermore, there is known an ultrasonic diagnostic system in which an ultrasonic image signal of a subject (patient) to be diagnosed is generated and stored in a cine memory, a camera image signal of the subject using a camera imaging device is generated and stored in the cine memory, and mixed frames of the ultrasonic image signal and the camera image signal are sorted and played in an order of time of arrival at the cine memory (see JP 2006-122078 A).

### SUMMARY

However, according to the ultrasonic diagnostic apparatus of JP 2009-207800 A, moving image data is not generated, though it is useful for medical treatment of a patient, rehabilitation, and the like. According to the ultrasonic diagnostic system of JP 2006-122078 A, moving image data is generated, but the operation for generating the ultrasonic image and the operation of the camera imaging device has to be performed separately, which puts large burden on an operator such as a doctor, a physiotherapist, or a technician.

An object of the present invention is to reduce the burden of operation regarding the ultrasonic image data and camera image data of a moving image.

To achieve at least one of the abovementioned objects, according to an aspect of the present invention, there is provided an ultrasonic diagnostic apparatus including:

an ultrasonic image generator that generates ultrasonic image data of a moving image based on a reception signal received from an ultrasonic probe that transmits and receives an ultrasonic wave to and from a subject,

a camera image generator that takes an image of an imaging target and generates camera image data of a moving image, and

a hardware processor that performs, in response to operation input related to ultrasonic diagnosis, processing related to the camera image data.

To achieve at least one of the abovementioned objects, according to another aspect of the present invention, there is provided an operating method of an ultrasonic diagnostic apparatus that comprises an ultrasonic image generator that generates ultrasonic image data of a moving image based on a reception signal received from an ultrasonic probe that transmits and receives an ultrasonic wave to and from a subject, and a camera image generator that takes an image of an imaging target and generates camera image data of a moving image, the operating method including:

in response to operation input related to ultrasonic diagnosis, controlling in which processing related to the camera image data is performed.

To achieve at least one of the abovementioned objects, according to another aspect of the present invention, there is provided a non-transitory computer-readable storage medium storing a program causing a computer to function as:

an ultrasonic image generator that generates ultrasonic image data of a moving image based on a reception signal received from an ultrasonic probe that transmits and receives an ultrasonic wave to and from a subject,

a camera image generator that takes an image of an imaging target and generates camera image data of a moving image, and

a hardware processor that performs, in response to operation input related to ultrasonic diagnosis, processing related to the camera image data.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a perspective view showing an outer appearance of an ultrasonic diagnostic apparatus according to an embodiment of the present invention;

FIG. 2 is a block diagram showing a functional configuration of the ultrasonic diagnostic apparatus;

FIG. 3 is a flowchart showing ultrasonic image generation processing;

FIG. 4 is a flowchart showing ultrasonic image generation processing following FIG. 3;

FIG. 5 is a flowchart showing ultrasonic image generation processing following FIG. 4;

FIG. 6 is a diagram showing a first image display screen;

FIG. 7 is a diagram showing a second image display screen;

FIG. 8 is a diagram showing a third image display screen;

FIG. 9 is a diagram showing a fourth image display screen; and

FIG. 10 is a flowchart showing ultrasonic image transfer processing.

### DETAILED DESCRIPTION OF THE EMBODIMENTS

Hereinafter, one or more embodiments and modifications of the present invention will be described with reference to the drawings. However, the scope of the invention is not limited to the illustrated examples.

### EMBODIMENTS

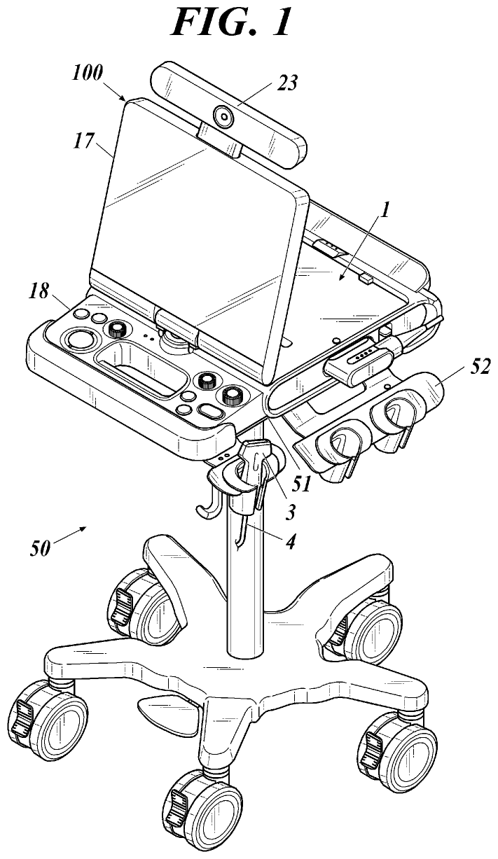

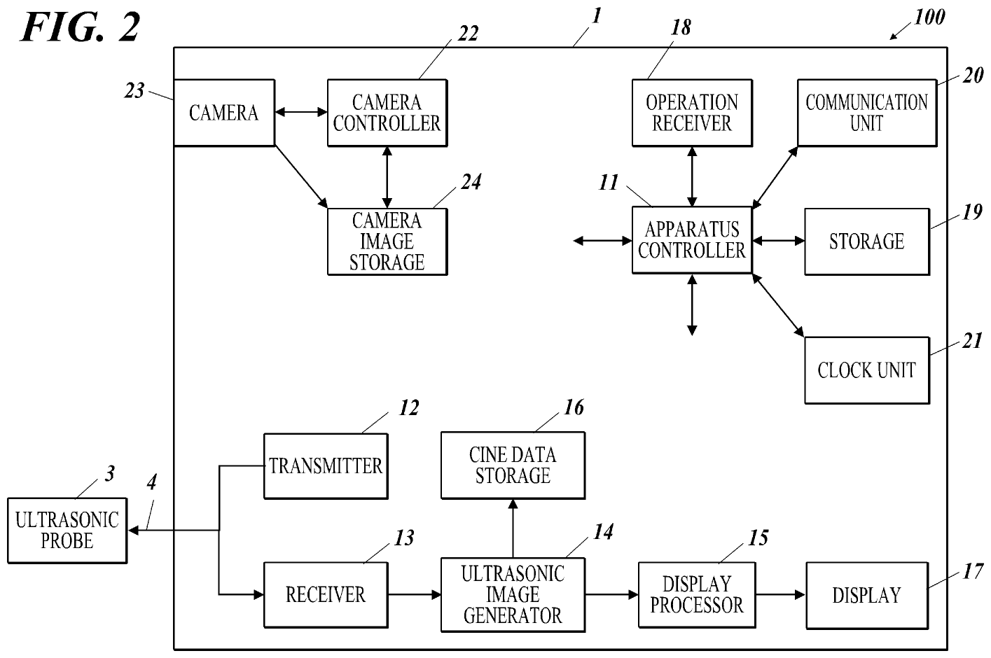

Referring to FIG. 1 to FIG. 10, an embodiment according to the present invention is described. First, referring to FIG. 1, the overall configuration of the apparatus of this embodiment will be described. FIG. 1 is a perspective view showing an outer appearance of an ultrasonic diagnostic apparatus 100. FIG. 2 is a block diagram showing a functional configuration of the ultrasonic diagnostic apparatus 100.

The ultrasonic diagnostic apparatus 100 of the present embodiment is an apparatus installed in a medical facility such as a hospital or a clinic, and generates an ultrasonic image of a subject such as a living body of a patient.

As shown in FIG. 2, the ultrasonic diagnostic apparatus 100 includes an ultrasonic diagnostic apparatus main body 1, an ultrasonic probe 3, and a cable 4. The ultrasonic probe 3 transmits transmission ultrasonic waves to the inside of the subject and receives reflected ultrasonic waves from the inside of the subject. The ultrasonic diagnostic apparatus main body 1 is connected to the ultrasonic probe 3 by the cable 4, transmits an electrical signal as a driving signal to the ultrasonic probe 3, and causes the ultrasound probe 3 to transmit transmission ultrasound waves to the subject. The ultrasonic probe 3 receives the reflected ultrasonic waves from the inside of the subject and, in response to the reflected ultrasonic waves, generates a reception signal that is an electrical signal. In response to the reception signal, the ultrasonic diagnostic apparatus main body 1 images an internal state of the subject as an ultrasonic image and generates ultrasonic image data.

The ultrasonic probe 3 has a transducer(s) (not shown in the drawings) that are piezoelectric elements. There are a plurality of transducers arranged in a one-dimensional array in the azimuth direction (scanning direction), for example. In the present embodiment, for example, the ultrasonic probe 3 includes 192 transducers. The transducers may be arranged in a two-dimensional array. The ultrasonic probe 3 may have any number of transducers. The ultrasonic probe 3 is a linear electronic scanning probe that scans ultrasonic waves by a linear scanning system in the present embodiment, but may be of any system such as a sector scanning system and a convex scanning system. The communication between the ultrasonic diagnostic apparatus main body 1 and the ultrasonic probe 3 may be performed wirelessly using Ultra Wide Band (UWB) and the like instead of by wire via the cable 4.

The ultrasonic diagnostic apparatus 100 is attached to a cart 50. The cart 50 is a trolley having wheels for moving. The cart 50 has an ultrasonic diagnostic apparatus attachment surface 51 and a holder 52. The ultrasonic diagnostic device attachment surface 51 is a flat surface to which the ultrasonic diagnostic apparatus main body 1 is attached. The holder 52 holds the ultrasonic probe 3.

As shown in FIG. 2, the ultrasonic diagnostic apparatus main body 1 includes, for example, an apparatus controller 11 as a controller (hardware processor), a transmitter 12, a receiver 13, an ultrasonic image generator 14, a display processor 15, a cine data storage 16, a display 17, an operation receiver 18, a storage 19, a communication unit 20, a clock unit 21, a camera controller 22, a camera 23 as a camera image generator, and a camera image storage 24.

The apparatus controller 11 includes, for example, a central processing unit (CPU), a read only memory (ROM), and a random access memory (RAM). The CPU reads various processing programs, such as a system program stored in the ROM, loads the processing programs to the RAM, and controls the operation of each unit of the ultrasonic diagnostic apparatus 100 according to the loaded programs. The ROM is configured by a non-volatile memory such as a semiconductor and stores a system program corresponding to the ultrasonic diagnostic apparatus 100, various processing programs executable on the system program, and various types of data such as a gamma table. These programs are stored in the form of computer-readable program code, and the CPU sequentially executes operations according to the program code. The RAM forms a work area for temporarily storing various programs executed by the CPU and data relevant to these programs.

The ROM of the apparatus controller 11 stores an image generation program for executing the image generation process described later and an ultrasonic image transfer program for executing the ultrasonic image transfer processing described later.

The transmitter 12 is a circuit that supplies a driving signal, which is an electrical signal, to the ultrasonic probe 3 under the control of the apparatus controller 11 and causes the ultrasonic probe 3 to generate transmission ultrasonic waves. The transmitter 12 includes, for example, a clock generating circuit, a delay circuit, a pulse generating circuit, or an arithmetic circuit that functions as these circuits. The clock generating circuit is a circuit that generates a clock signal for determining the transmission timing or the transmission frequency of the driving signal. The delay circuit is a circuit that sets a delay time of the transmission timing of the driving signal for each of the individual paths corresponding to the respective piezoelectric elements, delays the transmission of the driving signal by the set delay time, so as to focus the transmission beam formed by the transmission ultrasonic waves. The pulse generating circuit is a circuit for generating a pulse signal as a driving signal at a predetermined cycle. The transmitter 12 configured as described above generates ultrasonic waves by driving, for example, some successive transducers among the plurality of transducers arranged in the ultrasonic probe 3.

The receiver 13 is a circuit that receives a reception signal, which is an electrical signal, from the ultrasonic probe 3 under the control of the apparatus controller 11. The receiver 13 includes, for example, an amplifier, an A/D conversion circuit, and a phasing addition circuit. The amplifier is a circuit for amplifying the reception signal at a preset gain for each of the individual paths corresponding to the respective piezoelectric elements. The A/D conversion circuit is a circuit for A/D conversion of the amplified reception signal. The phasing addition circuit is a circuit that adjusts the time phase of the A/D-converted reception signal by giving a delay time for each of the individual paths corresponding to the respective piezoelectric elements, and adds up the time phases (performs phasing and addition) to generate sound ray data.

The ultrasonic image generator 14 generates B (Brightness) mode image data, which is tomographic image data as the ultrasonic image data, by performing envelope detection processing, log compression, and the like on the sound ray data from the receiver 13, adjusting the dynamic range and gain, and performing brightness conversion under the control of the apparatus controller 11. That is, the B mode image data represents the strength of the reception signal with the brightness. In addition to the B mode image data using the B mode, the ultrasonic image generator 14 may be one that can generate ultrasonic image data using another image mode, such as a color Doppler mode. Furthermore, the ultrasonic image generator 14 may perform various types of image processing on the generated ultrasonic image data. Furthermore, the ultrasonic image generator 14 stores the ultrasonic image data of a predetermined number of frames up to the latest frame in the cine data storage 16 as cine data.

Under the control of the apparatus controller 11, the display processor 15 converts the ultrasonic image data generated by the ultrasonic image generator 14 into an image signal for each frame, outputs it to the display 17, and displays it on the display 17.

The cine data storage 16 is composed of a RAM or the like, and temporarily stores ultrasonic image data of a plurality of predetermined frames input from the ultrasonic image generator 14 as cine data.

The applicable display 17 may be a display device such as an LCD (Liquid Crystal Display), an organic EL (Electronic Luminescence) display, an inorganic EL display, or a plasma display. The display 17 displays various types of display information such as an ultrasonic image on the display screen according to the image signal output from the display processor 15, the display information from the apparatus controller 11, and the like.

The operation receiver 18 includes various switches, various keys (hard keys), a trackball, a mouse, and the like to receive, for example, a command to start diagnosis, personal information data on a subject, and the like, and, in response to the operation input by an operator such as a doctor, a technician, or a physiotherapist, outputs an operation signal to the apparatus controller 11. The operation receiver 18 may include a touch panel formed on the display screen of the display 17, receive a touch operation from an inspector, and output the touch operation information to the apparatus controller 11.

The storage 19 is composed of, for example, a large-capacity recording medium such as an HDD (Hard Disk Drive) or an SSD (Solid State Drive), and stores data such as ultrasonic image data.

The communication unit 20 is composed of a network card or the like connected to a communication network (not shown), and transmits/receives information to/from a device(s) on the communication network. The apparatus controller 11 communicates with a device such as an image server (for example, a PACS (Picture Archiving and Communication Systems) server) (not shown) on the communication network via the communication unit 20

The clock unit 21 is a real-time clock, measures the current date and time, generates date and time information as current time information, and outputs the current time information to the apparatus controller 11

The camera controller 22 is a controller that controls the camera 23 and the camera image storage 24 under the control of the apparatus controller 11.

The camera 23 is, for example, a digital camera unit provided above the display 17 and capable of imaging (taking an image of) a subject. The camera 23 has an optical system, an imaging element, and the like, takes an image of an imaging target such as a subject (patient) as an imaging object, generates image data of a plurality of frames, and outputs the generated camera image data to the camera image storage 24 under the control of the camera controller 22. The camera 23 can change the frame rate (the number of frames generated per unit time, fps (flames per second)) in taking a moving image. The camera 23 (the camera controller 22, and the camera image storage 24) is (are) provided inside the ultrasonic diagnostic apparatus main body 1, but is not limited to this, and may be externally attached to the ultrasonic diagnostic apparatus main body 1.

The camera image storage 24 is composed of a RAM or the like, and temporarily stores image data of a plurality of predetermined frames input from the camera 23 as camera image data that is moving image data.

For each unit provided in the ultrasonic diagnostic apparatus 100, some or all of the functions of the respective functional blocks can be realized as a hardware circuit such as an integrated circuit. The integrated circuit is, for example, a large scale integration (LSI), and the LSI is called an integrated circuit (IC), a system LSI, a super LSI, or an ultra LSI, depending on the degree of integration. The method of circuit integration is not limited to LSI, and may be realized by a dedicated circuit or a general-purpose processor, or a field programmable gate array (FPGA) or a reconfigurable processor capable of reconfiguring the connection or setting of circuit cells inside the LSI may be used. Alternatively, the functions of some or all of the functional blocks may be executed by software. In this case, the software is stored in one or more storage media such as a ROM, an optical disk, or a hard disk, and is executed by an arithmetic processing device.

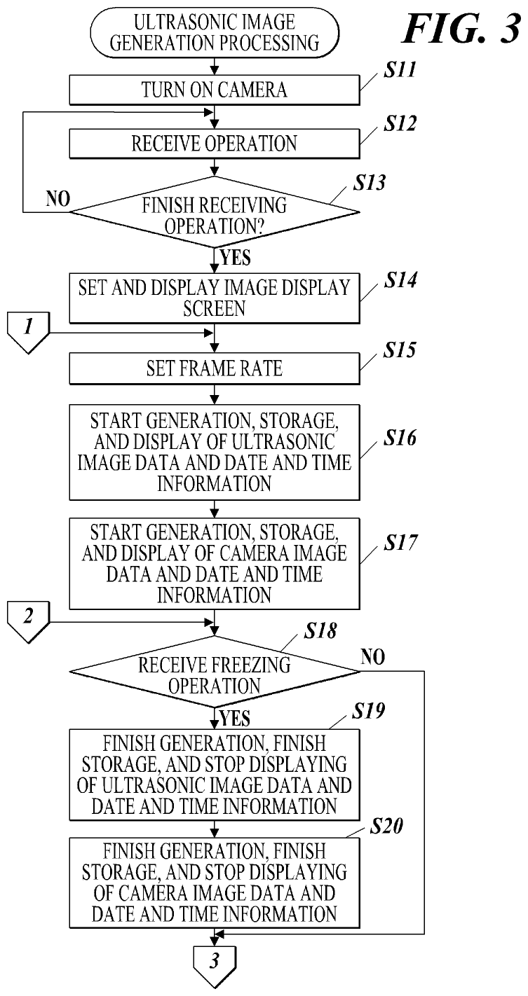

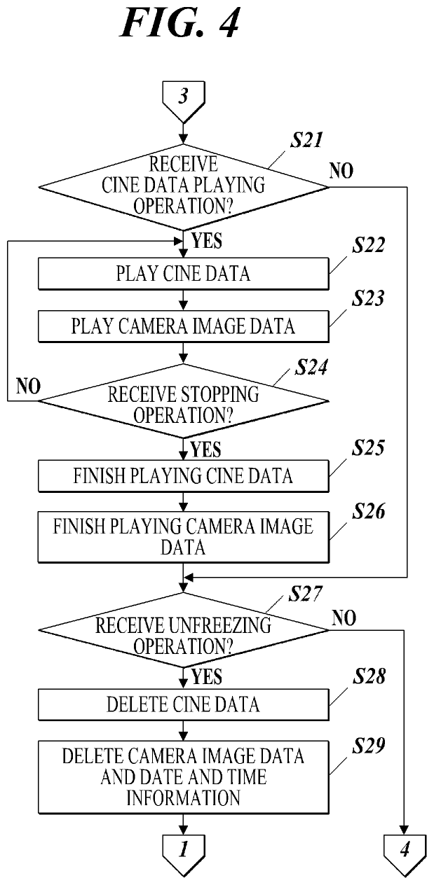

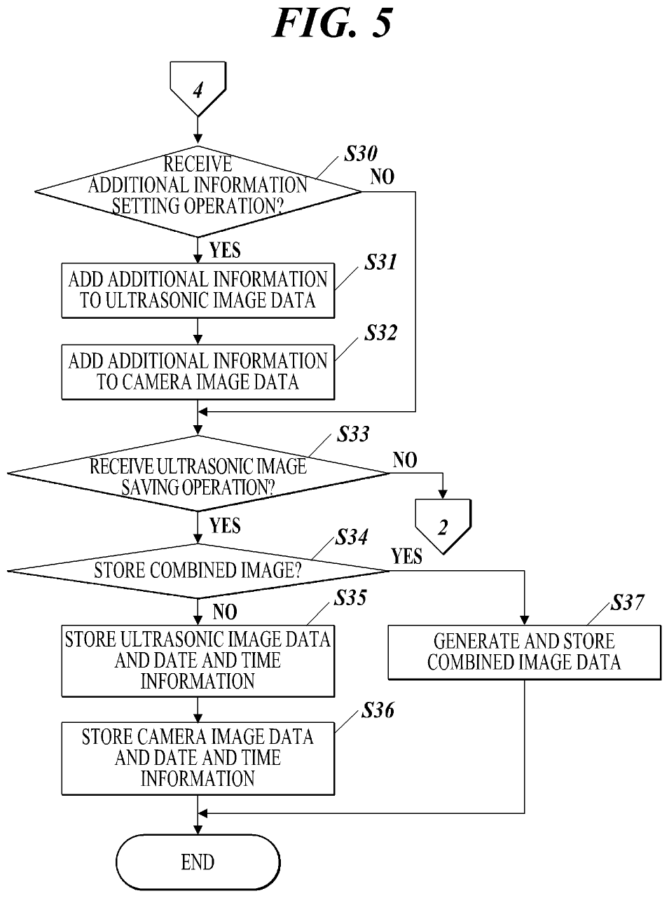

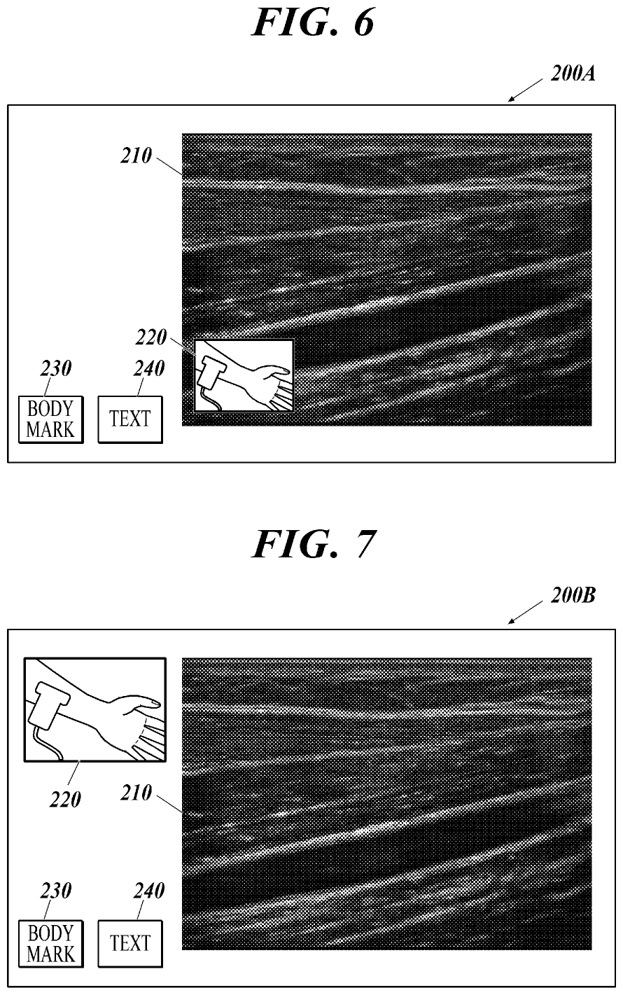

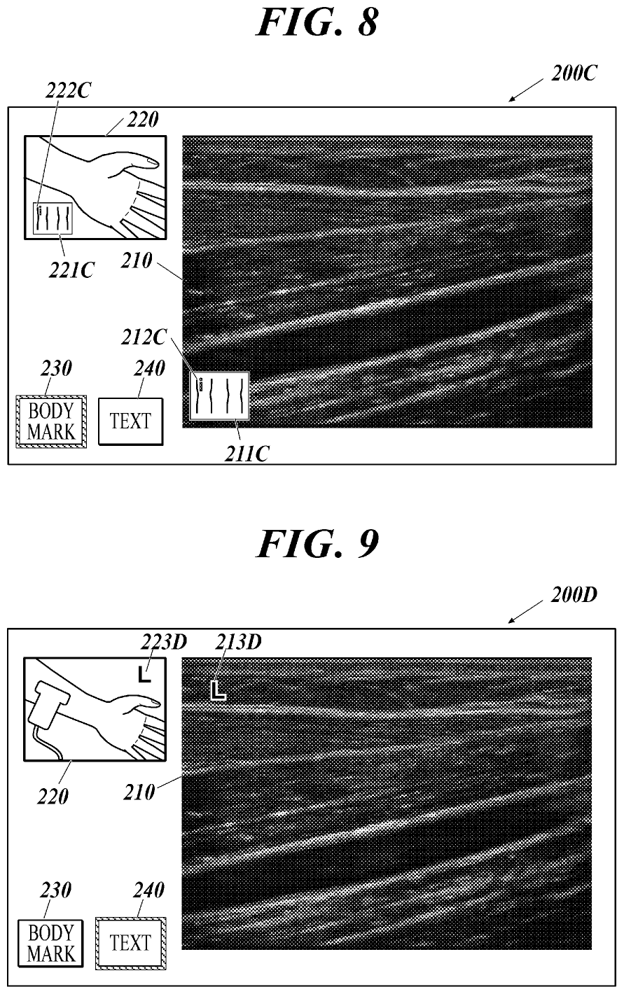

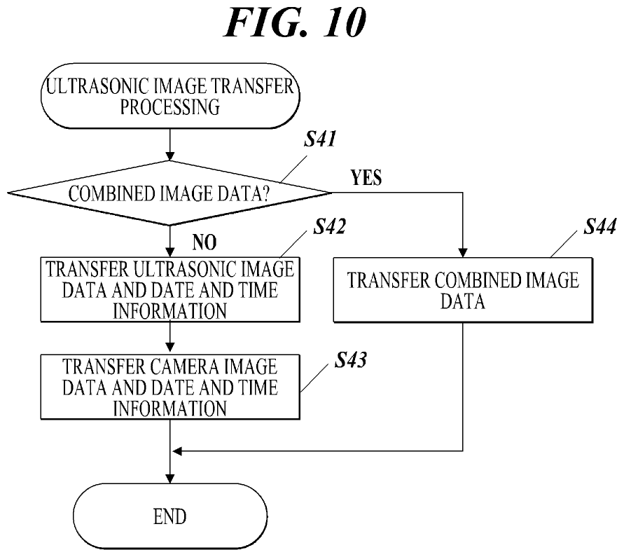

Next, the operation of the ultrasonic diagnostic apparatus 100 according to the present embodiment will be described with reference to FIG. 3 to FIG. 10. FIG. 3 is a flowchart illustrating ultrasonic image generation processing. FIG. 4 is a flowchart illustrating ultrasonic image generation processing following FIG. 3. FIG. 5 is a flowchart illustrating ultrasonic image generation processing following FIG. 4. FIG. 6 is a diagram showing an image display screen 200A. FIG. 7 is a diagram showing an image display screen 200B. FIG. 8 is a diagram showing an image display screen 200C. FIG. 9 is a diagram showing an image display screen 200D. FIG. 10 is a flowchart showing ultrasonic image transfer processing.

The ultrasonic image generation processing executed by the ultrasonic diagnostic apparatus 100 will be described with reference to FIG. 3 to FIG. 9. The ultrasonic image generation processing includes generating ultrasonic image data of a moving image by scanning an ultrasonic image of a subject, taking moving images of the subject in synchronization with the generation of the ultrasonic image data, generating camera image data of the moving image, and storing the camera image data.

In the case described below, in a medical facility, an operator such as a doctor and a patient who undergoes, for example, medical treatment or rehabilitation enter an examination room where the ultrasonic diagnostic apparatus 100 is set, and an ultrasonic image and a camera image of the patient are generated to be checked. The ultrasonic image is used to check an internal state of a predetermined portion of the patient as a subject, and the camera image is used to check an external state of the same.

In the ultrasonic diagnostic apparatus 100, triggered by turn on of the ultrasonic diagnostic apparatus 100 in response to the operator's operation input related to ultrasonic diagnosis, for example, the apparatus controller 11 executes the ultrasonic image generation processing in accordance with the ultrasonic image generation program stored in the ROM.

As shown in FIG. 3, first, the apparatus controller 11 turns on and activates the camera 23 (step S11). Then, the apparatus controller 11 receives, via the operation receiver 18, input including the operation information related to ultrasonic image data generation and the operation information related to camera image data generation as operation information on the operation input related to ultrasonic diagnosis by the operator (step S12). There may be no input of the operation information related to the camera image data generation.

The operation information in step S12 includes various types of setting information for ultrasonic image data generation (scanning) and various types of setting information for camera image data generation (taking images). The various types of setting information for generating the ultrasonic image data particularly in step S12 may include other information for generating the ultrasonic image data such as the frame rate of the ultrasonic image data to be generated, the setting values of various parameters (a sound ray density, a spatial compound, a time average, etc.) used for determining the frame rate of the ultrasonic image data, and setting values of Gain, Depth, and the like. The sound ray density is information that indicates density of sound rays of the transmitted ultrasonic waves emitted from the ultrasonic probe when the ultrasonic image data is generated. The spatial compound is information that indicates the number of combined ultrasonic images when the ultrasonic image data is generated, each based on sound rays in a corresponding direction. The time average is information that indicates processing intensity based on the number of ultrasonic images (for respective pixel values) for which the arithmetic mean or weighted average is taken in the ultrasonic image data generation, among the ultrasonic images of continuous frames. The operation information in step S12 may include the frame rate in generating the camera image. The setting information in step S12 may include setting information on the image display screen of the live or recorded ultrasonic image and the camera image. The operation information in step S12 may include text information such as patient ID, name, and examination ID input at the time of patient registration, and command information of starting examination. If some of the various types of the setting information is not input in step S12 but is required in steps S14, S15, S16, and S17, initial setting information of the various types of information stored in advance in the ROM of the apparatus controller 11 or the storage 19 is read out in step S12.

For example, as shown in FIG. 6, the setting information for displaying the image display screen 200A is input as the setting information of the image display screen. The image display screen 200A includes an ultrasonic image 210, a camera image 220, a body mark button 230, and a text button 240. The ultrasonic image 210 is an area in which an image of ultrasonic image data is displayed. The camera image 220 is an area in which an image of camera image data is displayed. The body mark button 230 is a trigger button for marking, with a body mark, the ultrasonic image data of the ultrasonic image 210 and the camera image data of the camera image 220. The text button 240 is a trigger button for marking, with a text such as an annotation, the ultrasonic image data of the ultrasonic image 210 and the camera image data of the camera image 220.

The operation information in step S12 may include, for example, information on the size and position of the ultrasonic image 210 and the camera image 220 on the image display screen 200A, and information on whether or not the camera image 220 is superimposed on the ultrasonic image 210. The image display screen 200A is an example of a screen in which the camera image 220 is superimposed on the ultrasonic image 210. The image display screen 200B shown in FIG. 6 is an example of a screen in which the camera image 220 is not superimposed on the ultrasonic image 210.

Then, the apparatus controller 11 determines whether or not the operation input in step S12 has been finished (step S13). If the operation input has not been finished (step S13; NO), the process proceeds to step S12. If the operation receiving has been finished (step S13; YES), the apparatus controller 11 generates an image display screen according to the setting information on the image display screen input in step S12 or the initial setting information set in advance, and displays the generated image display screen on the display 17 (step S14).

Then, the apparatus controller 11 sets the frame rate (first frame rate) for ultrasonic image data generation and the frame rate (second frame rate) for camera image data generation (step S15) according to the frame rate for ultrasonic image data generation input in step S12 or the parameter related to the frame rate, frame rate for camera image data generation, or the preset initial setting information. In step S15, the frame rate for the ultrasonic image data generation and the frame rate for the camera image data generation are matched so as to be the same as each other. For example, the frame rate of the image data generation set and input in step S12 is adjusted to the frame rate of the other image data generation.

Then, according to the operation information input in step S12 or the preset initial setting information and according to the frame rate of ultrasonic image data generation that is set in step S15, the apparatus controller 11 controls the transmitter 12, the receiver 13, the ultrasonic image generator 14, and the display processor 15 to start generation of ultrasonic image data, makes each frame of the ultrasonic image data correspond to the date and time information on the time of generation obtained from the clock unit 21, sequentially stores the generated ultrasonic image data and the date and time information as cine data in the cine data storage 16, and starts displaying a live ultrasonic image on the image display screen of the display 17 on the basis of the generated ultrasonic image data (step S16). For example, a live ultrasonic image is displayed on the ultrasonic image 210 on the image display screen 200A.

Then, according to the operation information input in step S12 or the preset initial setting information and according to the frame rate of camera image data generation that is set in step S15, the apparatus controller 11 controls the camera controller 22 and the camera 23 to start generation of camera image data, makes each frame of the camera image data correspond to the date and time information on the time of generation obtained from the clock unit 21, sequentially stores the generated camera image data and the date and time information in the camera image storage 24, and starts displaying a live camera image on the image display screen of the display 17 on the basis of the generated camera image data (step S17). For example, a live camera image is displayed on the camera image 220 on the image display screen 200A.

Then, the apparatus controller 11 determines whether or not the operator has input freezing operation of the live ultrasonic image as the operation input related to the ultrasonic diagnosis via the operation receiver 18 (step S18). If the freezing operation has been input (step S18; YES), the apparatus controller 11 controls the transmitter 12, the receiver 13, the ultrasonic image generator 14, and the display processor 15 to finish generation of the ultrasonic image data, to finish storage of the ultrasonic image data and the date and time information as cine data in the cine data storage 16, and to stop displaying of the live ultrasonic image on the display 17 (step S19).

Then, the apparatus controller 11 controls the camera controller 22 and the camera 23 to finish generation of the camera image data, to finish storage of the camera image data and the date and time information in the camera image storage 24, and to stop displaying of the live camera image on the display 17 (step S20).

Then, as shown in FIG. 4, the apparatus controller 11 determines whether or not the operator has input playing operation of the cine data during freezing as the operation input related to the ultrasonic diagnosis via the operation receiver 18 (step S21). If the freezing operation has not been input (step S18; NO), the process also proceeds to step S21. If the playing operation of the cine data has been input (step S21; YES), the apparatus controller 11 reads out the ultrasonic image data and the date and time information as the cine data from the cine data storage 16, and displays and plays, on the image display screen displayed in step S14, the ultrasonic image based on the ultrasonic image data according to the date and time information (step S22).

Then, the apparatus controller 11 reads out the camera image data and the date and time information from the camera image storage 24, and displays and plays the camera image based on the camera image data on the screen for playing displayed in step S14, in synchronization with the ultrasonic image data played in step S22 according to the date and time information (step S23).

Then, the apparatus controller 11 determines whether or not the operator has input stopping operation of playing the ultrasonic image as the operation input related to the ultrasonic diagnosis via the operation receiver 18 (step S24). If the stopping operation has not been input (step S24; NO), the process proceeds to step S22.

If the stopping operation has been input (step S24; YES), the apparatus controller 11 finishes playing of the ultrasonic image data in step S22 and stops (step S25). Then, at the same timing of the date and time information when the playing is finished in step S25, the apparatus controller 11 finishes playing of the camera image data in step S23 and stops (step S26).

In steps S21 to S26, the apparatus controller 11 plays and stops (finishes playing) the cine data of a moving image and the camera image data of a corresponding moving image on the display 17, but the present invention is not limited to this. An operator may input operation of selecting one frame of the cine data that is a moving image via the operation receiver 18, such that, in steps S21 to S26, the apparatus controller 11 plays (displays) the selected one frame, and plays (displays) one frame of the camera image data corresponding to the selected one frame on the display 17.

Then, the apparatus controller 11 determines whether or not the operator has input unfreezing operation during freezing, as the operation input related to the ultrasonic diagnosis via the operation receiver 18 (step S27). If the playing operation of the cine data has not been input (step S21; NO), the process also proceeds to step S27. If the unfreezing operation has been input (step S27; YES), the apparatus controller 11 deletes the ultrasonic image data and the date and time information as the cine data stored in the cine data storage 16 (step S28). Then, the apparatus controller 11 deletes the camera image data and the date and time information stored in the camera image storage 24 (step S29), and the process proceeds to step S15.

As shown in FIG. 5, if the unfreezing operation has not been input (step S27; NO), the apparatus controller 11 determines whether or not the operator has input setting operation of additional information to be added to the ultrasonic image data stored in the cine data storage 16 during freezing as the operation input related to the ultrasonic diagnosis via the operation receiver 18 (step S30).

In step S30, for example, as shown in the image display screen 200C of FIG. 8, in response to clicking operation of the body mark button 230, a body mark indicating the part of the subject selected from a body mark selection screen (not shown) and position information of the body mark are input as the additional information, and the position information of the probe mark indicating the position of the ultrasonic probe 3 to be included in the body mark is input. Furthermore, as shown in the image display screen 200D of FIG. 9, in response to clicking operation of the text button 240, text and its position information are input as additional information.

If additional information setting operation has been input (step S30; YES), the apparatus controller 11 sets addition of the additional information input in step S30 to the ultrasonic image data stored in the cine data storage 16 (step S31). Then, the apparatus controller 11 sets addition of the additional information input in step S30 to the camera image data stored in the camera image storage 24 (step S32).

In steps S31 and S32, for example, as shown in the image display screen 200C of FIG. 8, the body mark 211C of the imaging portion (arms) including the probe mark 212C indicating that the scan position is the left arm is added to the ultrasonic image data of the ultrasonic image 210, and the body mark 221C of the imaging portion (arms) including the probe mark 222C indicating that the scan position is the left arm is added to the camera image data of the camera image 220. By addition of the body mark and the probe mark, it is possible to check the camera image and the ultrasonic image at the scan position at the same time even when the position scanned by the ultrasonic probe 3 and the imaging position by the camera are different, for example, as in the camera image 220, in the case where a muscular layer of an arm is scanned to obtain an ultrasonic image while movement of fingers is observed in a camera image of the fingers.

In steps S31 and S32, for example, as shown in the image display screen 200D of FIG. 9, the text 213D of “L” indicating the imaging portion (left arm) is added to the ultrasonic image data of the ultrasonic image 210, and the text 223D of “L” indicating the imaging portion (left arm) is added to the camera image data of the camera image 210.

Then, the apparatus controller 11 determines whether or not the operator has input a command to store the ultrasonic image data as the operation input related to the ultrasonic diagnosis via the operation receiver 18 (step S33). If the additional information has not been input (step S30; NO), the process also proceeds to step S13. The storing command in step S33 includes information on whether or not to store the ultrasonic image data and the camera image data as combined image data, information on the size and position of the ultrasonic image and the camera image if they are combined, and information on whether to store ultrasonic image data of the plurality of frames as a moving image or to store the data of which one frame as a still image. If the command to store the ultrasonic image data has not been input (step S33; NO), the process proceeds to step S18.

If the command to store the ultrasonic image data has been input (step S33; YES), the apparatus controller 11 determines, according to the storing command in step S33, whether or not to store the combined image data (step S34).

If the combined image data is not stored (step S34; NO), according to the storing command in step S33, the apparatus controller 11 stores, as the moving image data in the storage 19, the ultrasonic image data of the plurality of frames and the date and time information stored in the cine data storage 16, or stores, as the still image data in the storage 19, the ultrasonic image data of one frame selected among the plurality of frames and the corresponding date and time information stored in the cine data storage 16 (step S35).

Then, in conjunction with and in the same manner as the storing of the ultrasonic image data, according to the storing command in step S33, the apparatus controller 11 stores, in the storage 19, the camera image data and the date and time information corresponding to the ultrasonic image data and the date and time information stored in step S35 (step S36). The camera image data is moving image data of a plurality of frames and the date and time information stored in the camera image storage 24, or still image data of one frame selected among the plurality of frames and the date and time information stored in the camera image storage 24. Then, the apparatus controller 11 finishes the ultrasonic image generation processing.

If combined image data is stored (step S34; YES), according to the storing command in step S33, the apparatus controller 11 stores combined image data as moving image data in the storage 19 or combined image data as still image data in the storage 19 (step S37). The combined image data as moving image data is generated by combining the ultrasonic image data of the plurality of frames stored in the cine data storage 16 and the camera image data of the plurality of frames stored in the camera image storage 24 using the date and time information, such that the frames of the same date and time information correspond to each other. The combined image data as still image data is generated by combining the ultrasonic image data of one selected frame stored in the cine data storage 16 and the camera image data of one frame of the same date and time information stored in the camera image storage 24. Then, the apparatus controller 11 finishes the ultrasonic image generation processing. The combined image data may be also stored so as to correspond to the date and time information of respective frames.

Because the stored ultrasonic image data and the camera image data each correspond to the date and time information, the camera image data can be played in synchronization with the ultrasonic image data using the date and time information, in the same manner as playing image data during freezing. Alternatively, the ultrasonic image and the camera image played in synchronization with each other by simply playing the stored combined image data.

Next, with reference to FIG. 10, the ultrasonic image transfer processing executed by the ultrasonic diagnostic apparatus 100 will be described. In the ultrasonic image transfer processing, the ultrasonic image data or the combined image data of the moving image generated and stored by the ultrasonic image generation processing is transferred to an external image server.

In the ultrasonic diagnostic apparatus 100, the apparatus controller 11 is triggered by input operation related to ultrasonic diagnosis by an operator such as a doctor, a technician, or a physiotherapist via the operation receiver 18 and executes the ultrasonic image transfer processing according to the ultrasonic image transfer program stored in the ROM. The input operation here is selection input of the ultrasonic image data or the combined image data stored in the storage 19 of the still image or the moving image as the transfer target.

First, the apparatus controller 11 determines whether or not the combined image data has been selected as the transfer target (step S41). If the ultrasonic image data has been selected (step S41; NO), the apparatus controller 11 reads out the ultrasonic image data that is selected for input and the date and time information from the storage 19, transfers the ultrasonic image data and the date and time information to the external image server via the communication unit 20, stores the ultrasonic image data and the date and time information in the external image server (step S42).

Then, according to the selectively input ultrasonic image data, the apparatus controller 11 reads out the camera image data of the still image or the moving image and the date and time information from the storage 19, transfers the camera image data and the date and time information via the communication unit 20 to the external image server, stores the camera image data and the date and time information in the external image server so as to correspond to the ultrasonic image data and the date and time information having been transferred in step S42 (step S43), and finishes the ultrasonic image transfer processing.

If the combined image data has been selected (step S41; YES), the apparatus controller 11 reads out the selectively input combined image data from the storage 19, and, via the communication unit 20, transfers it to an external image server, and stores it in the external image server (step S44), and finishes the ultrasonic image transfer processing.

As described above, according to the present embodiment, the ultrasonic diagnostic apparatus 100 includes the ultrasonic image generator 14 that generates ultrasonic image data of a moving image based on a reception signal received from the ultrasonic probe 3 that transmits and receives ultrasonic waves to and from a subject, the camera 23 that takes an image of an imaging target and generates camera image data of a moving image, and the apparatus controller 11 that performs processing related to the camera image data in accordance with the operation input related to the ultrasonic diagnosis. Therefore, the amount of operation related to the ultrasonic image data and the camera image data of the moving image can be reduced, the burden of operation can be reduced, and the usability can be improved.

Furthermore, the apparatus controller 11 performs processing related to the ultrasonic image data in response to an operation input related to the ultrasonic diagnosis, and performs processing related to camera image data in synchronization with the processing related to the ultrasonic image data. Therefore, the processing related to the ultrasonic image data can be performed in synchronization with the processing related to camera image data.

Furthermore, the processing related to the ultrasonic image data includes the process of starting and finishing generation of the ultrasonic image data by the ultrasonic image generator 14, and the processing related to the camera image data includes the process of starting and finishing generation of the camera image data by the camera 23. Therefore, it is possible to perform the process of starting and finishing the generation of the camera image data in conjunction with the operation input of the process of starting and finishing the generation of the ultrasonic image data, such that, for example, the efficiency of medical treatment and rehabilitation can be improved.

Furthermore, the processing related to the ultrasonic image data includes the process of playing the generated ultrasonic image data or starting and finishing play of the generated ultrasonic image data, and the processing related to the camera image data includes the process of playing the generated camera image data or starting and finishing play of the generated camera image data. Therefore, it is possible to play or to start and finish playing of the ultrasonic image data in conjunction with the process of playing or starting and finishing play of the generated camera image data. This can improve, for example, the efficiency of medical treatment and rehabilitation and can realize smooth communication between a doctor and a physiotherapist (understand the condition of diseases, grasp the position (muscle) to be stretched intensively, and the like) and smooth communication between a patient and a physiotherapist (visualization of the effects of rehabilitation). Furthermore, by playing the ultrasonic image data and the camera image data, it is possible to easily and reliably check the degree of the diseases and effects of rehabilitation (for example, to compare the healthy side and the affected side, to compare the image data recorded in the past with the current image data, and the like).

Furthermore, the processing related to the ultrasonic image data includes the process of storing the generated ultrasonic image data in the storage 19, and the processing related to the camera image data includes the process of storing the generated camera image data in the storage 19. Therefore, it is possible to perform the process of storing the camera image data in conjunction with the operation input of the process of storing the ultrasonic image data.

Furthermore, the processing related to the ultrasonic image data includes the process of transferring the generated ultrasonic image data to the image server as a transfer destination, and the processing related to the camera image data includes the process of transferring the camera image data to the image server. Therefore, it is possible to perform the process of transferring the camera image data in conjunction with the operation input of the process of transferring the ultrasonic image data.

Furthermore, the apparatus controller 11 causes each frame of the generated ultrasonic image data to correspond to the date and time information on generation, and causes each frame of the generated camera image data to correspond to the date and time information on generation. Therefore, it is possible to play the camera image data in synchronization with the playing of the ultrasonic image data easily by using the date and time information.

Furthermore, the apparatus controller 11 matches the first frame rate for generation/playing the ultrasonic image data and the second frame rate for generation/playing the camera image data. This makes it possible to easily play the ultrasonic image data and the camera image data in synchronization with each other.

Furthermore, the apparatus controller 11 adds additional information to the ultrasonic image data and the camera image data. Therefore, by using the additional information, it is possible to easily associate the ultrasonic image data and the camera image data, and by visually recognizing the additional information, it is possible to easily check the association.

Furthermore, the apparatus controller 11 combines the ultrasonic image data and the camera image data to generate the combined image data. Therefore, it is possible to reduce the size of image data including the ultrasonic image(s) and camera image(s), and to perform processes such as playing the image data easily.

### (Modifications)

Multiple modifications of the above embodiment will be described.

In the above embodiment, when the ultrasonic image data of the moving image and the camera image data of the moving image are generated in synchronization, the same additional information (body marks, texts, etc. in FIG. 8 and FIG. 9) is added to the ultrasonic image data and the camera image data. However, the present invention is not limited to this. The apparatus controller 11 may add additional information to the ultrasonic image data that is different from the additional information to the camera image data. For example, when the ultrasonic image data is generated and stored in synchronization with the camera image data, an annotation (text) indicating that there is synchronized camera image data may be added to the ultrasonic image data.

In the above embodiment, the method of synchronously generating and storing the ultrasonic image data of a moving image and the camera image data of a moving image includes matching the frame rates of the ultrasonic image data and the camera image data with each other, and further includes storing respective frames of the ultrasonic image data and the camera image data so as to correspond to the date and time information at the time of generation. However, the present invention is not limited to this.

For example, before generating the ultrasonic image data and the camera image data, the apparatus controller 11 may freely set the frame rate at the time of the generation. Then, the apparatus controller 11 generates the ultrasonic image data and the camera image data whose frames each correspond to the date and time information at the time of generation and stores the ultrasonic image data and the camera image data in the cine data storage 16 and the camera image storage 24. At the time of playing the ultrasonic image data, the apparatus controller 11 uses the date and time information corresponding to which each of the frames of the ultrasonic image data and the camera image data to adjust their play time, and plays each of the ultrasonic image data and the camera image data at the same frame rate or at different frame rates.

The apparatus controller 11 may freely set the frame rate (first frame rate and second frame rate, respectively) at the time of generating the ultrasonic image data and the camera image data and perform the generation and storage. Then, at the time of playing the ultrasonic image data and the camera image data, the apparatus controller 11 may play the ultrasonic image data and the camera image data at the frame rate of either of these. For example, at the time of playing the ultrasonic image data and the camera image data at a frame rate of image data generated at a larger frame rate, the apparatus controller 11 may interpolate frames of the image data generated at a smaller frame rate such that its frame rate is the same as the larger frame rate. The apparatus controller 11 may interpolate frames of camera image data, for example. Alternatively, at the time of playing the ultrasonic image data and the camera image data at a frame rate of image data generated at a smaller frame rate, the apparatus controller 11 may thin out frames of the image data generated at a larger frame rate such that its frame rate is the same as the smaller frame rate.

At the time of generating the ultrasonic image data and the camera image data, according to the image data at a larger frame rate, the apparatus controller 11 may interpolate frames of the other image data such that its frame rate is the same as the larger frame rate. Alternatively, at the time of generating the ultrasonic image data and the camera image data, according to the image data at a smaller frame rate, the apparatus controller 11 may thin out frames of the other image data such that its frame rate is the same as the smaller frame rate.

At the time of generating the ultrasonic image data and the camera image data, the apparatus controller 11 may set the frame rate of the generated camera image data (or the ultrasonic image data), such that the difference between the frame rates of the ultrasonic image data and the camera image data is decreased. For example, at the time of generating the ultrasonic image data and the camera image data, according to the image data at a larger frame rate, the apparatus controller 11 may interpolate frames of the other image data, such that its frame rate is less different from the larger frame rate. Alternatively, at the time of generating the ultrasonic image data and the camera image data, according to the image data at a smaller frame rate, the apparatus controller 11 may thin out frames of the other image data, such that its frame rate has less difference from the smaller frame rate.

At the time of playing the ultrasonic image data and the camera image data, the apparatus controller 11 may set the frame rate of the played camera image data (or the ultrasonic image data), such that the difference between the frame rates of the ultrasonic image data and the camera image data is decreased. For example, at the time of playing the ultrasonic image data and the camera image data, according to the image data at a larger one of these frame rates, the apparatus controller 11 may interpolate frames of the other image data, such that its frame rate is less different from the larger frame rate. Alternatively, at the time of playing the ultrasonic image data and the camera image data, according to one image data at a smaller one of these frame rates, the apparatus controller 11 may thin out frames of the other image data, such that its frame rate is less different from the smaller frame rate. As a result, it is possible to easily play the camera image data in synchronization with the playing of the ultrasonic image data.

At the time of generating the ultrasonic image data and the camera image data, the apparatus controller 11 may display preferable setting value(s) of various parameters (a sound ray density, a spatial compound, a time average, etc.) related to the frame rate of the ultrasonic image data generation on the display 17 and present them to the operator, such that the generated frame rate of the camera image data is the same as or less different from the generated frame rate of the ultrasonic image data. As a result, the operator can set the parameter(s) in generating the ultrasonic image data to the presented setting value(s) and thereby easily set the frame rate of the ultrasonic image data to one that is preferable for playing the camera image data in synchronization with the ultrasonic image data.

In the ultrasonic diagnostic apparatus 100, in parallel with the ultrasonic image generation processing, the apparatus controller 11 may perform image analysis of the live camera image data taken by the camera 23. When the apparatus controller 11 has recognized a predetermined posture of the subject (for example, a posture of a patient during predetermined rehabilitation), the apparatus controller 11 may perform control for playing a guidance image (for example, a moving image showing instructions on the treatment after the rehabilitation) on the display 17.

In the ultrasonic diagnostic apparatus 100, the apparatus controller 11 may perform image analysis of the live camera image data taken by the camera 23 and, when the apparatus controller 11 has recognized a predetermined posture of the subject (for example, a posture of a patient during rehabilitation), may display the guidance image and the live camera image data on the display 17 in synchronization with each other in terms of content. For example, a guidance image is displayed on the left side of the screen of the display unit 17, and a live camera image is displayed on the right side, and the cycle of raising/lowering operation of the arm is adjusted. When the operation speed is desired to be accelerated or decelerated, the playing speed of the guidance video may be adjusted depending on the playing speed of the live camera image. The guidance video is not limited to a moving image, but only audio data may be played. Alternatively, auxiliary information such as the direction of movement and the reference axis at the time of rehabilitation may be superimposed on the live camera image and displayed.

In the ultrasonic diagnostic apparatus 100, the apparatus controller 11 may perform image analysis of the live camera image data taken by the camera 23 and, when the apparatus controller 11 has recognized a predetermined movement or facial expression of the subject (for example, painful movements and facial expressions), may control generation of the ultrasonic image data or camera image data, or generation of the camera image data in synchronization with generation of the ultrasonic image data. The ultrasonic diagnostic apparatus 100 may have an audio receiver, and the apparatus controller 11 may perform audio analysis of the patient received by the audio receiver. When the apparatus controller 11 has recognized a predetermined voice of the subject (for example, a voice of a patient answering “five” in response to an orthopedist's question “Please rate your current pain on a scale of 1 to 10”), the apparatus controller 11 may control generation of the ultrasonic image data or camera image data, or generation of the camera image data in synchronization with generation of the ultrasonic image data. According to this configuration, the ultrasonic image data and the camera image data are generated without the operation of generating the ultrasonic image data by the operator, and the usability can be further improved.

In the ultrasonic diagnostic apparatus 100, the apparatus controller 11 may perform image analysis of the live camera image data taken by the camera 23 and, when the apparatus controller 11 has recognized a predetermined starting posture of the subject (for example, a posture in starting rehabilitation), may start generation and storage of the camera image data. When the apparatus controller 11 has recognized a predetermined finishing posture of the subject (for example, a posture in finishing rehabilitation), the apparatus controller 11 may finish generation and storage of the camera image data. The apparatus controller 11 can thereby generate the ultrasonic image data in synchronization with the camera image data generation from the start to the end of the camera image data generation. According to this configuration also, the ultrasonic image data and the camera image data are generated without the operation of generating the ultrasonic image data by the operator, and the usability can be further improved.

In the ultrasonic diagnostic apparatus 100, the apparatus controller 11 may quantitatively analyze a movement of the subject based on at least one of the generated camera image data and the ultrasonic image data, and may store the analysis information together with at least one of the camera image data and the ultrasonic image data in at least one of the cine data storage 16 and the camera image storage 24. For example, the apparatus controller 11 may generate analysis information by calculation of an angle between bones for image analysis of the subject's skeleton based on the camera image data, or by analysis of a moving distance of a point of interest for quantitation of the subject's movement based on the ultrasonic image data. According to such configurations, the analysis information can be stored and played together with at least one of the analysis sources, that is, the camera image data and the ultrasonic image data.

In the ultrasonic diagnostic apparatus 100, when the apparatus controller 11 receives, as the operation input related to the ultrasonic diagnosis via the operation receiver 18 by the operator, input of instructions on a finish of examination, a start of making a report, a finish of making a report, or the like, the apparatus controller 11 may control and finish the generation of the ultrasonic image data and the camera image data.

In the above ultrasonic diagnostic apparatus 100, the apparatus controller 11 controls the camera 23 in taking an image of the imaging target including the subject, but the imaging target may not include the subject. Only the ultrasonic probe 3 or a pricking needle may be taken as the imaging target.

The above-described embodiments and modifications merely disclose preferred examples of the ultrasonic diagnostic apparatus, the operating method of the ultrasonic diagnostic apparatus, and the storage medium according to the present invention, and the present invention is not limited thereto.

The detailed configuration and detailed operation of each part constituting the ultrasonic diagnostic apparatus 100 in the above embodiments and modifications can be appropriately changed without departing from the scope of the present invention. The scope of the present invention includes the scope of the present invention described in the scope of claims and the scope of their equivalents.

What is claimed is:

1. An ultrasonic diagnostic apparatus comprising:
an ultrasonic image generator that generates ultrasonic image data of a moving image based on a reception signal received from an ultrasonic probe that transmits and receives an ultrasonic wave to and from a subject,
a camera image generator that takes an image of an imaging target and generates camera image data of a moving image, and
a hardware processor that performs, in response to operation input related to ultrasonic diagnosis, processing related to the camera image data.

2. The ultrasonic diagnostic apparatus according to claim 1, wherein
the hardware processor performs, in response to the operation input related to ultrasonic diagnosis, processing related to the ultrasonic image data, and
the hardware processor performs the processing related to the camera image data in synchronization with the processing related to the ultrasonic image data.

3. The ultrasonic diagnostic apparatus according to claim 2, wherein,
in the processing related to the ultrasonic image data, the hardware processor causes the ultrasonic image generator to start and finish generation of ultrasonic image data, and
in the processing related to the camera image data, the hardware processor causes the camera image generator to start and finish generation of camera image data.

4. The ultrasonic diagnostic apparatus according to claim 2, wherein,
in the processing related to the ultrasonic image data, the hardware processor performs playing of the generated ultrasonic image data on a display or starts and finishes playing of generated the ultrasonic image data on the display, and,
in the processing related to the camera image data, the hardware processor performs playing of the generated camera image data on the display or starts and finishes playing of the generated camera image data on the display.

5. The ultrasonic diagnostic apparatus according to claim 2, wherein,
in the processing related to the ultrasonic image data, the hardware processor stores the generated ultrasonic image data in a storage, and
in the processing related to the camera image data, the hardware processor stores the generated camera image data in the storage.

6. The ultrasonic diagnostic apparatus according to claim 2, wherein,
in the processing related to the ultrasonic image data, the hardware processor transfers the generated ultrasonic image data to a transfer destination, and
in the processing related to the camera image data, the hardware processor transfers the generated camera image data to the transfer destination.

7. The ultrasonic diagnostic apparatus according to claim 1, wherein
the hardware processor makes each frame of the generated ultrasonic image data correspond to time information on generation time of the generated ultrasonic image data, and
the hardware processor makes each frame of the camera image data correspond to time information on generation time of the camera image data.

8. The ultrasonic diagnostic apparatus according to claim 1, wherein
the hardware processor performs at least one of generation or playing of the ultrasonic image data at a first frame rate,
the hardware processor performs at least one of generation or playing of the camera image data at a second frame rate, and
the hardware processor sets at least one of the first frame rate and the second frame rate, thereby making the first frame rate and the second frame rate correspond to or have a small difference between each other.

9. The ultrasonic diagnostic apparatus according to claim 8, wherein,
among the ultrasonic image data and the camera image data, the hardware processor interoperates a frame of image data that is generated or played at a smaller frame rate of the first frame rate and the second frame rate, thereby making the smaller frame rate correspond to or have a small difference from a larger frame rate of the first frame rate and the second frame rate.

10. The ultrasonic diagnostic apparatus according to claim 8, wherein,
among the ultrasonic image data and the camera image data, the hardware processor thins out a frame of image data that is generated or played at a larger frame rate of the first frame rate and the second frame rate, thereby making the larger frame rate correspond to or have a small difference from a smaller frame rate of the first frame rate and the second frame rate.

11. The ultrasonic diagnostic apparatus according to claim 8, wherein
the hardware processor presents a setting value of a parameter that is used in generation of the ultrasonic image data and that makes the first frame rate and the second frame rate correspond to or have a small difference between each other.

12. The ultrasonic diagnostic apparatus according to claim 11, wherein
the parameter includes at least one of a sound ray density, a spatial compound, and a time average.

13. The ultrasonic diagnostic apparatus according to claim 1, wherein
the hardware processor adds additional information to at least one of the ultrasonic image data and the camera image data.

14. The ultrasonic diagnostic apparatus according to claim 1, wherein
the hardware processor combines the ultrasonic image data and the camera image data, thereby generating combined image data.

15. An operating method of an ultrasonic diagnostic apparatus that comprises an ultrasonic image generator that generates ultrasonic image data of a moving image based on a reception signal received from an ultrasonic probe that transmits and receives an ultrasonic wave to and from a subject, and a camera image generator that takes an image of an imaging target and generates camera image data of a moving image, the operating method comprising:
in response to operation input related to ultrasonic diagnosis, controlling in which processing related to the camera image data is performed.

16. A non-transitory computer-readable storage medium storing a program causing a computer to function as:
an ultrasonic image generator that generates ultrasonic image data of a moving image based on a reception signal received from an ultrasonic probe that transmits and receives an ultrasonic wave to and from a subject,
a camera image generator that takes an image of an imaging target and generates camera image data of a moving image, and
a hardware processor that performs, in response to operation input related to ultrasonic diagnosis, processing related to the camera image data.
